(19) United States
(12) Patent Application Publication
(10) Pub. No.: US 2007/0237844 A1
(43) Pub. Date: Oct. 11, 2007

(54) Methods for Removing Contaminants from Essential Oils

(75) Inventors: Stephen G. Carlson, Altamonte Springs, FL (US); Esteban Arnoldo Bertera, Atlanta, GA (US); Theresa Sullivan Chamblee, Apopka, FL (US); Gregory John Murray, Mona Vale (AU); Ad Sidney Olansky, Decatur, GA (US); Terence Radford, Atlanta, GA (US)

Correspondence Address: SUTHERLAND ASBILL & BRENNAN LLP, 999 PEACHTREE STREET, N.E., ATLANTA, GA, 30309, US

(73) Assignee: The Coca-Cola Company, Atlanta, GA (US)

(21) Appl. No.: 11/279,028

(22) Filed: Apr. 7, 2006

(51) Int. Cl. A61K 36/752; A23L 1/22; A23L 2/56

(52) U.S. Cl. 424/736; 426/534

(57) ABSTRACT

Methods for removing a contaminant from an essential oil comprising contacting the essential oil including the contaminant with an aqueous alkaline solution.

### FIELD OF THE INVENTION

The present invention generally relates to methods for removing contaminants from essential oils.

### BACKGROUND OF THE INVENTION

Essential oils are volatile oils derived from fruit peels and leaves, stems, flowers, bark, roots, or twigs of plants, and usually carry the odor or flavor of the plant or its fruit. Essential oils are useful as flavorings for foods and beverages, as perfumes, and for medicinal purposes. For example, essential oils include peel oils such as citrus oil. Citrus oils are derived from squeezing or pressing citrus fruit peel. Citrus oils can be derived from lemons, oranges, limes, grapefruits, tangerines, mandarins, bitter oranges, and bergamots. Other essential oils include, but are not limited to leaf oils such as mint oils, spice oils such as clove oil, and flower oils such as rose oil.

Plants and fruits from which essential oils are derived are widely cultivated, in part for the essential oils they produce. During cultivation, agricultural chemicals are often applied to the plant or fruit, or both, to control pests such as insects, fungus, and weeds. While much of the agricultural chemicals applied during cultivation are removed by washing the harvest, some agricultural chemical residue sometimes remains on the plant or fruit from which essential oils are derived and can be extracted along with the essential oils. Furthermore, agricultural chemicals such as fungicides may be applied to harvested fruit to prevent spoilage and this fruit may be subsequently processed to produce essential oil. Thus, essential oils can sometimes contain trace amounts of agricultural chemicals which are referred to herein as agricultural residue.

It is therefore desirable to remove agricultural chemical residue from essential oils and there remains a need for an effective and economical method of removing such contaminants from essential oils.

### SUMMARY OF THE INVENTION

This invention addresses the foregoing need by providing methods for removing a contaminant from an essential oil comprising contacting the essential oil including the contaminant with an aqueous alkaline solution. Desirably, the aqueous alkaline solution removes the contaminant from the essential oil without substantially diminishing the organoleptic properties of the essential oil.

Other objects, features, and advantages of this invention will become apparent from the following description and claims.

### DETAILED DESCRIPTION OF EMBODIMENTS

As summarized above, this invention encompasses a method for removing contaminants from essential oils comprising contacting the essential oil including the contaminant with an aqueous alkaline solution. The aqueous alkaline solution desirably removes the contaminant without diminishing the organoleptic properties of the essential oil. Thus, the treated essential oil retains its desirable flavor or aroma, or both, but is more pure. In particular embodiments in which the essential oil is used in foods or beverages, removal of the contaminant is particularly desirable. Without wishing to be bound by theory, it is believed that, according to particular embodiments of the invention, the aqueous alkaline solution removes the contaminant from the essential oil either by (1) removing at least one proton from the contaminant to ionizethe contaminant and making it soluble in the aqueous alkaline solution or (2) converting the contaminant into a compound capable of donating a proton to the aqueous alkaline solution, wherein the converted contaminant may then undergo removal process (1). Thus, the contaminant or converted contaminant goes into solution in the aqueous alkaline solution and is thereby removed from the non-aqueous essential oil.

Essential oils treatable in accordance to the embodiments of this invention include all essential oils. Essential oils are volatile oils derived from fruit peels and leaves, stems, flowers, bark, roots, or twigs of plants, and usually carry the odor and/or flavor of the fruit or plant. Types of essential oils treatable in accordance with embodiments of this invention include, but are not limited to peel oils such as citrus oils, leaf oils such as mint oils, spice oils such as clove oil, flower oils such as rose oil, and other plant oil such as oil from stems, bark, roots and twigs. Citrus oils suitable for treatment in accordance with the embodiments of this invention include lemon oil, orange oil, lime oil, grapefruit oil, tangerine oil, mandarin oil, bitter orange oil, and bergamot oil. Embodiments of this invention are particularly effective in removing contaminants from citrus oils, but are also suitable for removing contaminants from any essential oil.

Embodiments of this invention are suitable for removing any essential oil contaminants that are removed by aqueous alkaline solutions. Examples of contaminants that are desirably removed from essential oils in accordance with embodiments of this invention include but are not limited to pesticides such as insecticides, fungicides, and herbicides. Some contaminants are neutral in nature, meaning that they are neither acidic nor alkaline in nature. Example of neutral pesticides removable in accordance with embodiments of the present invention include, but are not limited to, carbaryl. For example, carbaryl can be removed from the essential oil by contacting the essential oil with an aqueous alkaline solution to convert the carbaryl into 1-naphthol. The 1-naphthol is then ionized by the aqueous alkaline solution, which removes at least one proton from the 1-naphthol. The ionized 1-naphthol is soluble in the aqueous alkaline solution and thus, removal of the carbaryl and 1-naphthol from the essential oil is achieved.

Other contaminants are acidic in nature and are capable of donating at least one proton to an aqueous alkaline solution. Examples of pesticides which are acidic in nature and removable in accordance with the embodiments of this invention include, but are not limited to 1-naphthol and ortho-phenyl phenol (OPP). For instance, OPP can be removed from the essential oil by contacting the essential oil with an aqueous alkaline solution to remove at least one proton from the OPP. The removal of the proton from the OPP ionizes it and makes it soluble in the aqueous alkaline solution. Thus, removal of the OPP from the essential oil is as the ionized OPP goes into solution in the aqueous alkaline solution.

Other pesticides removable in accordance with embodiments of this invention include, but are not limited to 2,4-dichlorophenoxy acetic acid and 1-naphthol. The foregoing are merely examples of contaminants removable in accordance with embodiments of this invention, but embodiments of this invention are suitable to remove many other contaminants as well.

Suitable aqueous alkaline solutions, in accordance with embodiments of this invention, remove contaminants from essential oils while leaving the organoleptic properties of the essential oil alone. According to embodiments of this invention, suitable aqueous alkaline solutions include, but are not limited to, aqueous alkaline solutions comprising sodium hydroxide, potassium hydroxide, calcium hydroxide, magnesium hydroxide, and the like. Sodium hydroxide is a particularly suitable in an aqueous alkaline solution to remove carbaryl, OPP, 1-naphthol and other pesticides from essential oils which are to be used in consumer products because it is inexpensive and non-toxic in trace amounts. As would be known to those skilled in the art, other aqueous alkaline solutions which are characterized by a potency of hydroxyl ions in solution would be suitable for use with embodiments of this invention. In particular embodiments, the aqueous alkaline solution has a pH greater than 7. In other embodiments, the aqueous alkaline solution has a pH between about 13 and 14.

According to particular embodiments of the invention, the aqueous alkaline solution comprises sodium hydroxide and water and the sodium hydroxide is present in the aqueous alkaline solution in an amount ranging from about 0.1 grams of sodium hydroxide for each 100 milliliters of water present in the aqueous alkaline solution to about 50 grams of sodium hydroxide for each 100 milliliters of water present in the aqueous alkaline solution. In other embodiments, the aqueous alkaline solution comprises sodium hydroxide and water and the sodium hydroxide is present in the aqueous alkaline solution in an amount ranging from about 1 gram of sodium hydroxide for each 100 milliliters of water present in the aqueous alkaline solution to about 7 grams of sodium hydroxide for each 100 milliliters of water present in the aqueous alkaline solution.

Essential oils are treated with aqueous alkaline solutions in accordance with conventional methods such as combining the essential oil including the contaminant and the aqueous alkaline solution in a chamber to constitute an aqueous phase and an oil phase. This contacting of the essential oil with the aqueous alkaline solution desirably removes at least a portion of the contaminant. Preferably, the contaminant is substantially completely removed from the essential oil.

Desirably, the relative amounts of aqueous alkaline solution and essential oil are such that the desired removal of contaminant is achieved. According to particular embodiments of the invention, the essential oil and the aqueous alkaline solution may be combined in an essential oil to aqueous alkaline solution ratio based on parts by weight of the essential oil to parts by weight of the aqueous alkaline solution ranging from about 1:1 to about 50:1. In other embodiments, the essential oil and the aqueous alkaline solution may be combined in an essential oil to aqueous alkaline solution ratio based on parts by weight of the essential oil to parts by weight of the aqueous alkaline solution ranging from about 1:1 to about 20:1. However, it should be understood by a person of ordinary skill in the art that, in addition to the essential oil to aqueous alkaline solution ratio, the desired removal of contaminant may be dependnt upon the concentration of hydroxide in the aqueous alkaline solution, the contact time of the essential oil to the aqueous alkaline solution, the degree of contact achieved between the essential oil and the aqueous alkaline solution, the temperature of the essential oil and alkaline solution, or the concentration of the contaminant in the essential oil, among other factors.

Particular embodiments of the invention may be useful in treating essential oils including contaminant in concentrations of up to 500 PPM. In other particular embodiments, essential oils including contaminants in concentrations between 0.01 PPM and 100 PPM contacted with an aqueous alkaline solution to remove the contaminants.

According to particular embodiments of the invention, a method is provided for removing the contaminant from the essential oil by combining the essential oil and the aqueous alkaline solution in a chamber to constitute an aqueous phase and an oil phase. The aqueous phase comprises the aqueous alkaline solution, which upon contact with the essential oil, then includes the contaminant. The organic phase comprises the essential oil, which is then substantially contaminant free. The organic phase can then be separated from the aqueous phase by any method of separation of organic and aqueous phases, as is well known to those skilled in the art.

In other embodiments, the greater contact of the essential oil with the aqueous alkaline solution can be achieved by agitating the essential oil and the aqueous alkaline solution in a chamber for an agitation time. Since such agitation forms an emulsion in which the essential oil and the aqueous alkaline solution are blended, embodiments of the invention may further include resting the essential oil and aqueous alkaline solution for a resting time to form an aqueous phase and an oil phase after agitating the essential oil and the aqueous alkaline solution. Once the aqueous phase and oil phase are formed, the two can be separated from one another. In particular embodiments, the agitation time may be greater than 1 hour.

According to particular embodiments of the invention, an inert gas may be introduced into a chamber in which the essential oil and the aqueous alkaline solution are being combined. The inert gas can be used to purge at least a portion of any gaseous oxidizing agents which may be present in the chamber. This purge of any oxidizing agents prevents oxidation of the essential oil, which may affect the organoleptic properties of the essential oil. Examples of suitable inert gases for embodiments of this invention include, but are not limited to, nitrogen and argon.

In particular embodiments, any residual contaminants and/or alkali may be removed from the essential oil which was contacted with and then separated from the aqueous alkaline solution by performing a water-wash of the essential oil. The water-wash comprises contacting the essential oil with water, which causes any residual contaminants and/or alkali to go into solution in the water and then separating the water from the essential oil, thereby removing the residual contaminants and/or the alkali. The detailed mechanism of this water-wash is not explained in further detail, as such specifics are understood by a person of ordinary skill in the art.

In alternate embodiments, the essential oil may exhibit cloudiness after being contacted with the aqueous alkaline solution to remove the contaminant. In such embodiments, filtering or centrifuging of the essential oil can be performed to remove the opaque material.

For example, in an embodiment in which essential oil containing OPP is treated, a 5% aqueous solution of NaOH or a 7% solution of KOH is combined in a container such as a separatory funnel with the essential oil, such as grapefruit or lemon oil, in a 1:1 weight ratio. The combination is shaken for 45 seconds to 1 minute and then allowed to stand so that the aqueous solution and oil separate in the funnel. The aqueous solution is the drawn off the bottom of the funnel. The essential oil is then washed by adding an equal volume of water to the oil, shaking the combination, allowing the oil and water to separate and then drawing off the water. When OPP is the contaminant, the reaction is very quick and can cloud the oil. The oil can be filtered or centrifuged to remove the cloudiness. 95 to 97% of the OPP can be removed in such a process.

The present invention is further illustrated below in an example describing removal of carbaryl. This example is not to be construed in any way as imposing limitations upon the scope of the invention. On the contrary, it is to be clearly understood that resort may be had to various other embodiments, modifications, and equivalents thereof which, after reading the description therein, may suggest themselves to those skilled in the art without departing from the scope of the invention and the appended claims.

### EXAMPLE 1

An example of an embodiment of a method for removing a contaminant from an essential oil by contacting the essential oil including the contaminant with an aqueous alkaline solution in accordance with embodiments of the present invention is disclosed in this example.

An essential oil comprising 105.81 grams of cold-pressed orange oil included carbaryl at 15.51 PPM. The orange oil was contacted with 5.57 grams of an aqueous alkaline solution comprising a 5% w/w sodium hydroxide solution in an Erlenmeyer flask containing a magnetic stirring bar. The flask headspace was filled with nitrogen. The contents were stirred vigorously for 2 hours at room temperature. Next, the orange oil and sodium hydroxide solution were allowed to separate. The oil phase was then filtered with a #3 Whatman filter. Finally, the orange oil was water-washed. Separation of the water-wash from the oil occurred over a period of one to two days. Analysis of the orange oil resulted in no carbaryl or 1-naphthol being detected.

As explained above, the mechanism for removal of carbaryl is believed to be different from the mechanism for removing OPP. As a result, removal of carbaryl takes longer, but carbaryl can be removed to a point that its presence is not detectable.

It should be understood that the foregoing relates to particular embodiments of the present invention and that numerous changes may be made therein without departing from the scope of the invention as defined by the following claims.

We claim:

1. Method for removing a contaminant from an essential oil comprising contacting the essential oil including the contaminant with an aqueous alkaline solution.

2. Method as in claim 1 wherein the contaminant is a neutral organic compound.

3. Method as in claim 1 wherein the contaminant is an organic compound which is capable of donating at least one proton to the aqueous alkaline solution.

4. Method as in claim 2 wherein the contaminant is a neutral organic compound and the aqueous alkaline solution converts the neutral organic compound into an organic compound which is capable of donating at least one proton to the aqueous alkaline solution.

5. Method as in claim 1 wherein the contaminant is carbaryl.

6. Method as in claim 4 wherein the neutral organic compound is carbaryl and the organic compound which is capable of donating at least one proton to the aqueous alkaline solution is 1-naphthol.

7. Method as in claim 1 wherein the contaminant is ortho-phenyl phenol or 1-naphthol.

8. Method as in claim 1 wherein the essential oil is selected from the group consisting of peel oil, leaf oil, spice oil, and flower oil.

9. Method as in claim 1 wherein the essential oil comprises citrus oil.

10. Method as in claim 9 wherein the citrus oil is selected from the group consisting of lemon oil, orange oil, lime oil, grapefruit oil, mandarin oil, bitter orange oil, and bergamot oil.

11. Method as in claim 1 wherein the aqueous alkaline solution comprises a hydroxyl ion source chosen from the group consisting of sodium hydroxide, potassium hydroxide, calcium hydroxide, and magnesium hydroxide.

12. Method as in claim 1 wherein the aqueous alkaline solution comprises sodium hydroxide and water, and wherein the sodium hydroxide is present in the aqueous alkaline solution in an amount ranging from about 0.1 grams of sodium hydroxide for each 100 milliliters of water present in the aqueous alkaline solution to about 50 grams of sodium hydroxide for each 100 milliliters of water present in the aqueous alkaline solution.

13. Method as in claim 1 wherein the aqueous alkaline solution comprises sodium hydroxide and water, and wherein the sodium hydroxide is present in the aqueous alkaline solution in an amount ranging from about 1 grams of sodium hydroxide for each 100 milliliters of water present in the aqueous alkaline solution to about 7 grams of sodium hydroxide for each 100 milliliters of water present in the aqueous alkaline solution.

14. Method as in claim 1 wherein the aqueous alkaline solution has a pH greater than 7.

15. Method as in claim 1 wherein the aqueous alkaline solution has a pH between about 13 and about 14.

16. Method as in claim 1 wherein the contaminant is an agricultural residue.

17. Method as in claim 16 wherein the agricultural residue is a pesticide.

18. Method as in claim 16 wherein the contaminant is selected from the group consisting of carbaryl, ortho-phenyl phenol, 1-naphthol, and 2,4-dichlorophenoxy acetic acid.

19. Method as in claim 1 wherein the step of contacting the essential oil with the aqueous alkaline solution comprises combining the essential oil and the aqueous alkaline solution in a chamber to form an aqueous phase and an oil phase.

20. Method as in claim 19 further comprising separating the aqueous phase from the oil phase.

21. Method as in claim 19 wherein the step of contacting the essential oil with the aqueous alkaline solution comprises combining the essential oil and the aqueous alkaline solution in a chamber in an essential oil to aqueous alkaline solution ratio based on parts by weight of the essential oil to parts by weight of the aqueous alkaline solution ranging from about 1:1 to about 50:1.

22. Method as in claim 19 wherein the step of contacting the essential oil with the aqueous alkaline solution comprises combining the essential oil and the aqueous alkaline solution in a chamber in an essential oil to aqueous alkaline solution ratio based on parts by weight of the essential oil to parts by weight of the aqueous alkaline solution ranging from about 1:1 to about 20:1.

23. Method as in claim 1 wherein the step of contacting the essential oil with the aqueous alkaline solution comprises agitating the essential oil and the aqueous alkaline solution in a chamber for an agitation time, and wherein the method further comprises resting the essential oil and aqueous alkaline solution for a resting time to form an aqueous phase and an oil phase after the step of agitating.

24. Method as in claim 23 wherein the agitation time is greater than 1 hour.

25. Method as in claim 23 further comprising separating the aqueous phase from the oil phase.

26. Method as in claim 23 further comprising introducing an inert gas into the chamber, wherein the inert gas purges at least a portion of any gaseous oxidizing agents from the chamber.

27. Method as in claim 25 further comprising contacting the oil phase with water after the step of separating the aqueous phase from the oil phase and then separating the water from the oil phase.

28. Method for removing a contaminant from an essential oil comprising contacting the essential oil including the contaminant with an aqueous alkaline solution such that the aqueous alkaline solution removes at least a portion of the contaminant without substantially diminishing the organoleptic properties of the essential oil.

29. Method as in claim 28 wherein the aqueous alkaline solution substantially entirely removes the contaminant from the essential oil.

30. Method as in claim 28 wherein the contaminant is a neutral organic compound.

31. Method as in claim 28 wherein the contaminant is an organic compound which is capable of donating at least one proton to the aqueous alkaline solution.

32. Method as in claim 28 wherein the contaminant is a neutral organic compound and the aqueous alkaline solution converts the neutral organic compound into an organic compound which is capable of donating at least one proton to the aqueous alkaline solution.

33. Method as in claim 28 wherein the contaminant is carbaryl.

34. Method as in claim 32 wherein the neutral organic compound is carbaryl and the organic compound which is capable of donating at least one proton is 1-naphthol.

35. Method as in claim 28 wherein the contaminant is ortho-phenyl phenol or 1-naphthol.

36. Method as in claim 28 wherein the essential oil comprises citrus oil.

37. Method as in claim 28 wherein the citrus oil is selected from the group consisting of lemon oil, orange oil, lime oil, grapefruit oil, mandarin oil, bitter orange oil, and bergamot oil.
